(19) United States
(12) Patent Application Publication
(10) Pub. No.: US 2010/0267957 A1
(43) Pub. Date: Oct. 21, 2010

(54) Anthraquinone Dyes As Photosensitizers In Photovoltaic Cells

(75) Inventor: Friedrich Lehr, Efringen-Kirchen (DE)

Correspondence Address: CLARIANT CORPORATION;INTELLECTUAL PROPERTY DEPARTMENT, 4000 MONROE ROAD, CHARLOTTE, NC, 28205, US

(73) Assignee: CLARIANT FINANCE (BVI) LIMITED, Tortola (VG)

(21) Appl. No.: 12/742,671

(22) PCT Filed: Nov. 13, 2008
(86) PCT No.: PCT/EP08/65498

§ 371 Date: May 13, 2010

(30) Foreign Application Priority Data
Nov. 16, 2007 (EP) 07120918.3
Jan. 9, 2008 (EP) 08150134.8
Nov. 13, 2008 (EP) PCT/EP2008/065498

(51) Int. Cl. C07D 221/18; C07C 211/45; C07C 211/54; C07C 49/675

(52) U.S. Cl. 546/76; 564/427; 568/326

(57) ABSTRACT

The use of anthraquinone, anthrone, anthrimide or anthrapyridone as a photosensitizer dye in a metal oxide layer of a dye-sensitized photochemical solar cell.

The invention relates to the use of anthraquinone, anthrone, anthrimide or anthrapyrimidine dyestuffs in photovoltaic cells. These dyes can be coated on titanium dioxide films rendering the device effective in the conversion of visible light to electrical energy.

Titanium dioxide and other transition metal oxides films (layers) are known for their semiconductive properties and this property renders them useful for photovoltaic cells. It is important that the titanium dioxide film is coated with a In Close contact with a photosensitizer such films convert light to electricity, preferably in range of the solar spectrum in the wavelength domain where the sun emits light, i.e., between 300 and 2000 nm.

Dye-sensitized photochemical solar cells are known from e.g.
“Dye-sensitized regenerative solar cells”; McEvoy, Augustin J.; Graetzel, Michael (Ecole Polytechnique Federale de Lausanne, Lausanne, Switzerland). Encyclopedia of Electrochemistry, 2003, 6, 397-406 (Eng). Edited by Bard, Allen J.; Stratmann, Martin. Wiley-VCH Verlag GmbH & Co. KG & Weinheim, Germany; ISBN 3-527-30398-7 or
“Dyes for semiconductor sensitization.”; Nazeeruddin, Md. Khaja; Graetzel, Michael (Swiss Federal Institute of Technology, Lausanne, Switz.). Encyclopedia of Electrochemistry) 2003, 6, 407-431 (Eng). Edited by Bard, Allen J.; Stratmann, Martin. Wiley-VCH Verlag GmbH & Co. KG & Weinheim, Germany; ISBN 3-527-30398-7 or
“Dye-sensitized solar cells.”; Kmon, J. M.; O'Regan, B. C.; van Roosmalen, J. A. M.; Sinke, W. C. (Solar Energy, Energy Research Centre of the Netherlands, 1755 ZG Petten, Neth.) in Handbook of Photochemistry and Photobiology 2003, 1, 1-47 (Eng). Edited by Nalwa, Hari Singh. American Scientific Publishers: Stevenson Ranch, Calif. 91381-1439, USA; ISBN: 1-58883-004-7 or
“Dye-sensitized photoelectrochemical solar cells.”; Iha, Neyde Yukie M u r m Garcia, Christian Graziani; Bignozzi, Carlo A. (Institute de Quimica, Universidade de Sao Paulo, 05508-900 Sao Paulo, Brazil). In Handbook of Photochemistry and Photobiology 2003, 1, 49-82 (Eng). Edited by Nalwa, Han Singh. American Scientific Publishers: Stevenson Ranch, Calif. 91381-1439, USA; ISBN: 1-58883-004-7 and the references cited in these Articles.

However, there is still a need for improved sensitizing dyes in dye-sensitized photochemical solar cells.

According to the invention there is provided a photovoltaic cell comprising:
a light transmitting electrically conductive layer deposited on a glass plate or a transparent polymer sheet to which have been applied one or more metal oxide layers (hereinafter referred to as “metal oxide layers”), the metal oxide being selected from titanium dioxide (e.g., anatase and rutile), titanates (e.g., sodium, barium or strontium titanates), niobates (e.g., potassium niobate), tin oxide, iron oxide, zinc oxide, indium oxide, bismuth oxide, Bismuth vanadate zirconium dioxide, yttrium trioxide (Y₂O₃), tungsten trioxide and molybdenum trioxide to mixtures of said metal oxide layers, to the uppermost layer of which a photosensitizer dye has been applied, such a photosensitizer being an anthraquinone, anthrone, anthrimide or anthrapyridone dye, (herein defined as the photosensitizer) characterized in that the photosensitizer dye selected from one or more compounds selected from compounds of formula I to VI as described below.

The invention relates to a dye-sensitized photochemical solar cell comprising dyes of the formula I, II, III. IV, V or VI

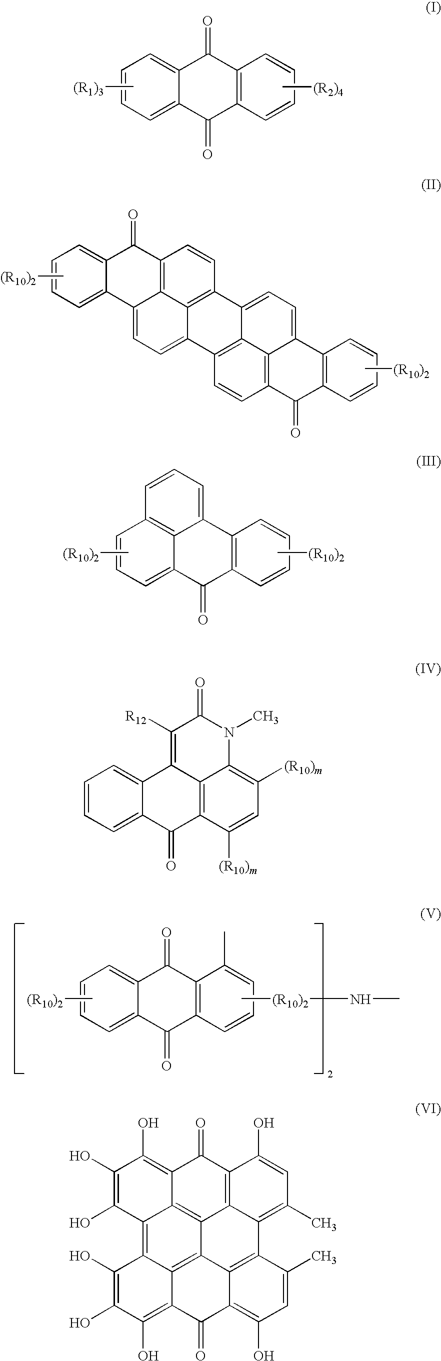

in which each R₁ independently is selected from hydrogen, —NH₂, —SO₃H, —SH, C₁₋₈alkyl, —OH, —COOH, halogen, —NHC₁₋₄alkyl, —NH(CH₂)₁₋₂COOH, —NHCOR₃, —NHOH, —NHCH₂(CH₂)₁₋₂OH, —N(C₁₋₄alkyl)₂,

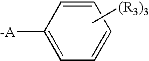

—OC₁₋₄alkyl, —OCH₂(CH₂)₁₋₂—COOH and —OCH₂(CH₂)₁₋₃—OH;
each group R₂ has a significance of R₁, independent of R₁, provided that at least one group R₂ is hydrogen or two groups R₂ are ortho to one another and have a significance of R₁ (preferably OH) and the other two groups R₂ are ortho to one another and form a group α or β

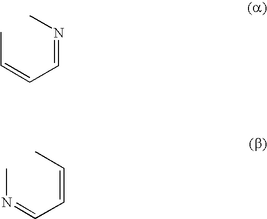

R₃ is selected from halogen C₁₋₄alkyl, —COOH, NH₂, OH and hydrogen.

### A is —NH— or —O—;

R₁₀ is hydrogen, —NH₂, —OH, SH, —CO₂R₁₂, C₁₋₈alkyl, —(CH₂)₁₋₂—CO₂R₁₂, —NHR₁₂, —NR₁₂, —OR₁₂, —SR₁₂ wherein R₁₂ is hydrogen or C₁₋₈alkyl;
and m is 0 or 1
with the proviso that 1,2-dihydroxyanthra-9,10-chinone, 1,2,4-trihydroxyanthra-9,10-chinone and Isoviolanthrone are excluded from the scope of protection

The invention further relates to a dye-sensitized photochemical solar cell comprising dyes of the formula I, II, III. IV, V or VI as sensitizing dyes

The invention further relates to the use of dyes of the formula I, II, III. IV, V or VI as sensitizing dyes in dye-sensitized photochemical solar cells.

Preferred compounds of formulae Ito VI are of formula I′

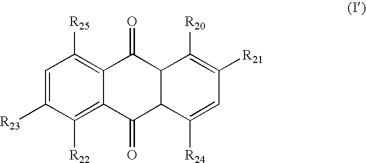

in which each of R₂₀ to R₂₅ independently is selected from hydrogen —NH₂, OH, C₁₋₈alkyl,

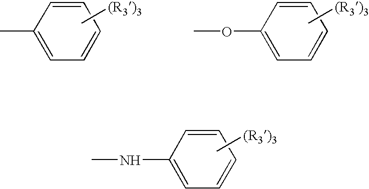

wherein R₃′ is hydrogen or C₁₋₄alkyl.

The C₁₋₈alkyl preferably is tert-butyl.

More preferably each of R₂₀ to R₂₅ is hydrogen, —OH or —NH₂.

Most preferably R₂₀ is —OH or —NH₂, R₂₁ is OH or NH₂ and R₂₂ is —NH₂ or hydrogen and R₂₃ is hydrogen or OH and R₂₄ and R₂₅ are independently OH or hydrogen preferably R₂₄ and R₂₅ are hydrogen.

Preferably the metal oxide is titanium dioxide.

For example, the transparent conductive layer used in a photovoltaic cell according to the invention is made of tin dioxide doped with ca 0.8 atom percent of fluorine and this layer is deposited on a transparent substrate made of low cost soda lime float glass. This type of conducting glass can be obtained from Asahi Glass Company, Ltd. Tokyo, Japan. under the brand name of TCO glass. The transparent conductive layer can also be made of indium oxide doped with up to 5% tin oxide, deposited on a glass substrate. This is available from Balzers under the brand name of ITO glass.

By selecting appropriate dyestuffs, the cell can be optimized with respect to solar energy conversion. A photovoltaic cell according to the present invention has an optimal threshold wavelength for light absorption at 820 nm corresponding to an energy of 1.5 eV. Such a cell can attain higher solar conversion efficiencies than a cell based on silicon.

It is preferable that only the last three, the last two or just the very top layer of the metal oxide layers is doped with a divalent or trivalent metal in an amount of not more than 15% doping.

All of the metal oxide layers are formed by the sol-gel process method described above. Preferably the number of metal oxide layers deposited is 10-11. Preferably the total thickness of the metal oxide film is from 5 to 50 microns (more preferably 10-20 microns).

Further according to the invention there is provided an electrode comprising a transparent metal oxide layer on a glass support, for use in photovoltaic cell systems, to which the Photosensitizer has been applied.

Preferably this metal oxide layer is produced by dispersion of colloidal TiO₂ solutions on glass support. Preferably such solutions are prepared by hydrolysis of Ti(OCH(CH₃)₂)₄. Preferably such TiO₂ layers are transparent.

Preferably the Photosensitizer is bond or coordinated to metal atoms. The bonding may be of physical or chemical nature. Preference is given to charge-transfer complexes. Charge-transfer complexes are combinations of electron donor compounds with electron acceptor compounds. The charge-transfer complexes are assembled in defined stacks. More preferred are Photosensitizer coordinated to metal atoms. The Photosensitizer coordinated to metal atoms by at least one covalent bond via the O— or N— atoms of the Photosensitizer, more preferably the Photosensitizer is bond to the metal atoms by two or more of the O— or N— atoms. The Photosensitizer, when bond by several covalent bonds to the metal atoms, maybe bond to the same metal atom or to several different, e.g. two or more, metal atoms.

In addition the photovoltaic cell of the present invention may contain other chemical additives designed to provide specific properties. These include co-adsorbents, surfactants, gelators, ionic liquids, etc.

By the term “transparent” is meant that 70%, more preferably 80% of incident light passes through the glass.

Compounds of formula I to VI are known and can be made by known methods.

The invention will now be illustrated by the following Examples.

### EXAMPLES

### Example 1

A photovoltaic device based on the sensitization of an aluminum doped titanium dioxide membrane supported on conducting glass is fabricated as follows:

A stock solution of the organic titanium dioxide precursor is prepared by dissolving 21 mmol of freshly distilled TiCl₄ in 10 ml of absolute ethanol. The stock solution is then diluted to give a titanium content of 25 mg/ml (solution A) or 50 mg/ml (solution B). A third solution (C) is prepared from solution B by addition of the appropriate quantity of AlCl₃ to yield an aluminum content of 1.25 mg/ml. A conducting glass sheet provided by Asahi Inc. Japan, surface area 10 cm², optical transmission in the visible at least 85%, surface resistance smaller than 10 ohms per square cm is used as support for the TiO₂ layer. Prior to use, the glass is cleaned with alcohol. A droplet of solution A is spread over the surface of the conducting glass to produce a thin coating. Subsequently the titanium alkoxide layer is hydrolyzed at 28° C. for 30 minutes in a special chamber where the humidity is kept at 48% of the equilibrium saturation pressure of water. Thereafter, the electrode is heated in air in a tubular oven kept at 450° C., preheating it in the entrance of the oven for 5 minutes followed by 15 minutes of heating in the interior. Three more layers are produced in the same way. Subsequently, 5 thicker layers are deposited by using solution B. The same procedure as for the first layers is applied. Finally, solution C is used to deposit the last two layers containing the aluminum dope. The heating of the last layer in the tubular oven was extended from 15 to 30 minutes. The total thickness of the titanium dioxide film is between 10 and 20 microns.

Prior to deposition of the dye, the film is subjected to a sintering treatment in highly purified 99.997% argon. A horizontal tubular oven composed of quartz tubes with suitable joints is employed. After insertion of the glass sheet loaded with TiO₂, the tube is twice evacuated and purged with argon. The glass supported TiO₂ layer is then heated under argon flux at a rate of (2.5 L/h) 500° C./h up to 550° C. at which temperature it maintained for 35 minutes. This treatment produces anatase films with a surface roughness factor of 80-200.

After cooling the glass supported TiO₂ layer under a continuous argon flow, it is immediately transferred to an ethanolic solution of the dye No. 1 of Table 1.

Its concentration in absolute ethanol is 5×10⁴M. Prolonged exposure of the film to the open air prior to dye adsorption is avoided in order to prevent hydroxylation of the TiO₂ surface. The presence of hydroxyl groups at the electrode surface interferes with dye uptake. The adsorption of dye from the ethanolic solution is allowed to continue for 30 minutes after which time the glass sheet is withdrawn and washed briefly with absolute ethanol. The TiO₂ overlayer on the sheet assumed a deep color owing to the dye coating.

A photovoltaic cell, shown in FIG. 1, is constructed, using the dye (4) loaded TiO₂ (5) film supported on the conducting glass (the working electrode) comprising the conductive tin dioxide layer (6) and the glass substrate (7) as a photoanode. The cell has a sandwich like configuration, the working electrode (4 to 7) being separated from the counter electrode (1,2) by a thin layer of electrolyte (3) having a thickness of ca 20 microns. The electrolyte was an ethanolic solution of 0.5M Lil and 3×10⁻³M iodine. The electrolyte (3) is contained in a small cylindrical reservoir (not shown) attached to the side of the cell from where capillary forces attract it to the inter-electrode space. The counter electrode was made also of Asahi conducting glass. The conductive tin dioxide layer (2) deposited on a glass substrate (1) is placed directly on top of the working electrode. A monomolecular transparent layer of platinum is deposited onto the conducting glass of the counter electrode (1,2) by electroplating from an aqueous hexachloroplatinate solution. The role of the platinum is to enhance the electrochemical reduction of iodine at the counter electrode. The transparent nature of the counter electrode is an advantage for photovoltaic applications since it allows the harvesting of light from both the forward and the backward direction. Experiments are carried out with a high pressure Xenon lamp equipped with appropriate filters to simulate AM1 solar radiation. The intensity of the light is varied between 50 and 910 Watts per square meter and the open circuit voltage is 660 and 800 mV, respectively at these two voltages. The fill factor defined as the maximum electric power output of the cell divided by the product of open circuit current and short circuit voltage is given in Table 2 below. A single crystal silicon cell gave an open voltage of 550 mV at 600 W/m² incident light intensity which dropped to below 300 mV at 50 W/m2. This clearly shows that the cell of the present invention has a higher open circuit voltage than the silicon solar cell and that the open circuit voltage is less dependent on light intensity than that of the silicon cell. This constitutes a significant advantage for the use of such a cell in indirect sunlight or cloudy weather conditions. The fill factor of the silicon cell is comparable to that of the example.

TABLE 1

| No | R¹ | R² | R³ | R⁴ |
|---|---|---|---|---|
| 1. | NH₂ | OH | | |
| 2. | NH₂ | OH | | |
| 3. | OH | OH | | |
| 4. | OH | OH | | |
| 5. | OH | OH | | |
| 6. | OH | OH | | |
| 7. | NH₂ | OH | | OH |
| 8. | NH₂ | | | |
| 9. | 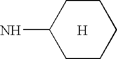 | | | 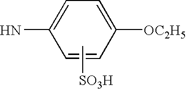 |
| 10. | NH₂ | | | OH |
| 11. | NH₂ | Br | Br | OH |
| 12. | NH₂ | | Br | OH |
| 13. | 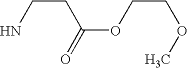 | | | OH |
| 14. | NH₂ | CO₂H | | |
| 15. | NH₂ | | | |
| 16. | | NH₂ | | |
| 17. | NH₂ | NH₂ | | |
| 18. | NH₂ | | | NH₂ |
| 19. | NH₂ | | | |
| 20. | NH₂ | | | NH₂ |
| 21. | | NH₂ | | |
| 22. | | NH₂ | | |
| 23. | NH₂ | SO₃H | | |
| 24. | NH₂ | CH₃ | | |
| 25. | NH₂ | OH | | SO₃H |
| 26. | NH₂ | SO₃H | | SO₃H |
| 27. | NH₂ | | | OH |
| 28. | NH₂ | Cl | | |
| 29. | NHCH₃ | | | |
| 30. | NHOH | | | |
| 31. | NHOH | Cl | | |
| 32. | OH | | | |
| 33. | OH | | | NH₂ |
| 34. | OH | | | |
| 35. | | SO₃H | | |
| 36. | OH | | | |
| 37. | OH | | | OH |
| 38. | SO₃H | | | |
| 39. | OH | OH | | OH |

| 40. | Isoviolanthrone | | | |
|---|---|---|---|---|
| 41. | OH | OH | COOH | OH |
| 42. | NH(CH)₂COOH | | | OH |
| 43. | NH₂ | | | NH₂ |
| 44. | | COOH | | |
| 45. | Benzanthrone | | | |
| 46. | OH | OH | OH | |
| 47. | OH | | OH | |
| 48. | OH | OH | | OH |
| 49. | | NHCOCH₃ | COOH | |
| 50. | OH | OH | | |
| 51. | indigo-anil | | | |
| 52. | | COOH | | |
| 53. | NH₂ | | | |
| 54. | NH₂ | | | |
| 55. | | NH₂ | COOH | |
| 56. | NH₂ | SO₃H | | NHCO(C₆H₄-o-COOH) |
| 57. | NH₂ | SO₃H | | NHCO(C₆H₄-o-COOH) |
| 58. | NHCO(C₆H₅) | | | |
| 59. | NH₂ | | | NH(C₆H₅) |
| 60. | OH | | | OH |
| 61. | NO₂ | | | |
| 62. | SH | NH₂ | | |
| 63. | NO₂ | | | |
| 64. | NO₂ | | | |
| 65. | Dianthrimid | | | |
| 66. | NHCH₃ | | | NH(C₆H₄-o-COOH) |
| 67. | OH | OH | | |
| 68. | NH₂ | | | NH(C₆H₅) |
| 69. | NH₂ | | | |
| 70. | NH₂ | | | NHCO(C₆H₅) |
| 71. | NH₂ | SH | | |
| 72. | NHCO(C₆H₅) | | | |
| 73. | | NH₂ | NH₂ | |
| 74. | NH₂ | | | NH₂ |
| 75. | NHC₆H₅ | | | |
| 76. | NHC₆H₅ | | | |
| 77. | NH-cyclohexyl | | | |
| 78. | NHCH₃ | | | |
| 79. | NHCH₃ | | | p-NHC₆H₄NH₂ |
| 80. | p-NHC₆H₄NH₂ | | | |
| 81. | (N-methyl-2-OH-(1,9)-anthrapyridone) | | | |
| 82. | NHC₆H₅ | | | NHCOC₆H₄-p-NH₂ |
| 83. | NHC₆H₄-p-NH₂ | | | |
| 84. | NH-cyclohexyl | | | NH-cyclohexyl |
| 85. | NHC₆H₅ | | | |
| 86. | NH₂ | | | NHCH₃ |
| 87. | NHCH₂CH₂OH | | | NHCH₂CH₂OH |
| 88. | OH | | | NHCOC₆H₅ |
| 89. | N(CH₃)₂ | | | N(CH₃)₂ |
| 90. | NH₂ | | | |
| 91. | NH₂ | | | NH₂ |
| 92. | OH | | | OH |
| 93. | NHCH₂CH₂OH | | | NHCH₂CH₂OH |
| 94. | OH | | | NHCOC₆H₅ |
| 95. | N(CH₃)₂ | | | N(CH₃)₂ |
| 96. | NH₂ | | | NHCOC₆H₅ |
| 97. | NH₂ | | | |
| 98. | NH₂ | | | NH₂ |
| 99. | OH | | | OH |
| 100. | | | | OH |
| 101. | OH | OH | | OH |
| 102. | OH | OH | | |
| 103. | OH | OH | | 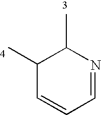 |
| 104. | NH₂ | OH | | |
| 105. | OH | OH | | 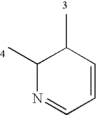 |
| 106. | NH₂ | OH | | |
| 107. | OH | OH | | |
| 108. | OH | OH | | |
| 109. | NH₂ | OH | | |
| 110. | OH | OH | | 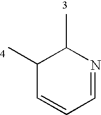 |
| 111. | NH₂ | OH | | |
| 112. | NH₂ | OH | | |
| 113. | OH | OH | | OH |
| 114. | OH | OH | OH | OH |
| 115. | OH | OH | OH | |
| 116. | OH | | OH | |
| 117. | OH | OH | | p-NHC₆H₄CH₃ |
| 118. | NH₂ | SO₃H | | p-NHC₆H₄NHCOCH₃ |
| 119. | NH₂ | SO₃H | | 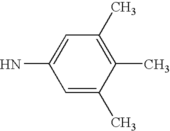 |
| 120. | NH₂ | SO₃H | | 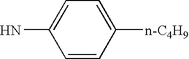 |
| 121. | NHC₆H₁₁ | | | 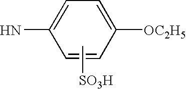 |
| 122. | NH₂ | SO₃H | | 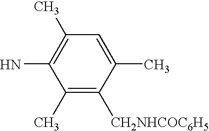 |
| 123. | NH₂ | SO₃H | | 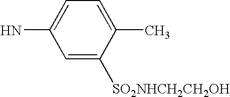 |
| 124. | NH₂ | SO₃H | | |
| 125. | NH₂ | Br | | 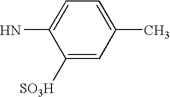 |
| 126. | NH₂ | | | NH₂ |
| 127. | OH | | | NHC₆H₄ |
| 128. | | | CN | NH₂ |
| 129. | OH | | | p-NHC₆H₄CH₂CH₂OH |
| 130. | NH₂ | | Br | OH |
| 131. | NH₂ | | Br | OH |
| 132. | NH₂ | SO₃H | | 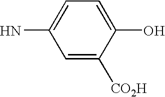 |
| 133. | NH₂ | | | 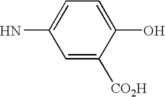 |
| 134. | NH₂ | | SO₃H | OH |
| 135. | NH₂ | | SO₃H | OH |
| 136. | NH₂ | | CO₂H | OH |
| 137. | NH₂ | COCH₃ | | |
| 138. | OH | NH₂ | | |
| 139. | NH₂ | | | |
| 140. | NH₂ | CO₂H | | |
| 141. | NH₂ | | | OH |
| 142. | NH₂ | CO₂H | | OH |
| 143. | NH₂ | SH | | OH |
| 144. | NH₂ | SH | | NH₂ |
| 145. | NH₂ | NHC₆H₄-p-OH | | |
| 146. | NH₂ | OCH₃ | | NH₂ |
| 147. | NH₂ | SO₃H | | NH₂ |
| 148. | NH₂ | C₆H₄-p-OH | | OH |
| 149. | NH₂ | OH | OCH₃ | NHC₆H₅ |
| 150. | OH | OH | OCH₃ | NHC₆H₅ |
| 151. | NH₂ | OH | CH(C₆H₄N(CH₃)₂)₂ | N(CH₃)₂ |
| 152. | NH₂ | OH | | N(CH₃)₂ |
| 153. | NH₂ | OH | | NHCOC₆H₅ |
| 154. | NH₂ | OH | | |
| 155. | NH₂ | OH | 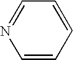 | 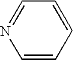 |
| 156. | NH₂ | SO₃Na | | Br |

| No. | R⁵ | R⁶ | R⁷ | R⁸ | R⁹ | R¹⁰ |
|---|---|---|---|---|---|---|
| 1. | | | | | ═O | ═O |
| 2. | NH₂ | OH | | | ═O | ═O |
| 3. | | | | | ═O | ═O |
| 4. | OH | OH | | | ═O | ═O |
| 5. | | OH | | | ═O | ═O |
| 6. | | OH | | OH | ═O | ═O |
| 7. | | | | | ═O | ═O |
| 8. | NH₂ | | | | ═O | ═O |
| 9. | | | | | ═O | ═O |
| 10. | NH₂ | | | OH | ═O | ═O |
| 11. | NH₂ | | | OH | ═O | ═O |
| 12. | OH | | | NH₂ | ═O | ═O |
| 13. | 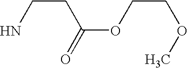 | | | OH | ═O | ═O |
| 14. | NH₂ | | | | ═O | ═O |
| 15. | | | | | ═O | ═O |
| 16. | | | | | ═O | ═O |
| 17. | | | | | ═O | ═O |
| 18. | | | | | ═O | ═O |
| 19. | | | | NH₂ | ═O | ═O |
| 20. | NH₂ | | | NH₂ | ═O | ═O |
| 21. | | | NH₂ | | ═O | ═O |
| 22. | | | NH₂ | | ═O | ═O |
| 23. | | | | NH₂ | ═O | ═O |
| 24. | | | | | ═O | ═O |
| 25. | | | | | ═O | ═O |
| 26. | | | | | ═O | ═O |
| 27. | | | | | ═O | ═O |
| 28. | NH₂ | Cl | | | ═O | ═O |
| 29. | | | | | ═O | ═O |
| 30. | | | | | ═O | ═O |
| 31. | NHOH | Cl | | | ═O | ═O |
| 32. | | | | | ═O | ═O |
| 33. | OH | | | NH₂ | ═O | ═O |
| 34. | OH | | | | ═O | ═O |
| 35. | SO₃H | | | | ═O | ═O |
| 36. | | | | OH | ═O | ═O |
| 37. | | | | | OH | OH |
| 38. | | | | SO₃H | ═O | ═O |
| 39. | | | | | ═O | ═O |
| 40. | | | | | | |
| 41. | | | | | ═O | ═O |
| 42. | | | | | ═O | ═O |
| 43. | | | | | OH | OH |
| 44. | | | | | ═O | ═O |
| 45. | | | | | | |
| 46. | OH | OH | OH | | ═O | ═O |
| 47. | | | | | ═O | ═O |
| 48. | | | | | ═O | ═O |
| 49. | | | | | ═O | ═O |
| 50. | | OH | | | ═O | ═O |
| 51. | | | | | | |
| 52. | | | | | ═O | ═O |
| 53. | OH | | | | ═O | ═O |
| 54. | | COOH | | | ═O | ═O |
| 55. | | | | | ═O | ═O |
| 56. | | | | | ═O | ═O |
| 57. | | | | | ═O | ═O |
| 58. | NHCO(C₆H₅) | | | | ═O | ═O |
| 59. | | | | | ═O | ═O |
| 60. | | | | | OH | OH |
| 61. | | 6(7)COOH | | | ═O | ═O |
| 62. | | | | | ═O | ═O |
| 63. | | COOH | | | ═O | ═O |
| 64. | | | | COOH | ═O | ═O |
| 65. | | | | | | |
| 66. | | | | | ═O | ═O |
| 67. | | | OH | | ═O | ═O |
| 68. | | | | | ═O | ═O |
| 69. | NHCOC₆H₅ | | | | ═O | ═O |
| 70. | | | | | ═O | ═O |
| 71. | | | | | ═O | ═O |
| 72. | | | | | ═O | ═O |
| 73. | | | | | ═O | ═O |
| 74. | NHC₆H₅ | | | | ═O | ═O |
| 75. | NHC₆H₅ | | | | ═O | ═O |
| 76. | | | | NHC₆H₅ | ═O | ═O |
| 77. | NH-cyclohexyl | | | | ═O | ═O |
| 78. | NHCH₃ | | | | ═O | ═O |
| 79. | | | | | ═O | ═O |
| 80. | | | | | ═O | ═O |
| 81. | | | | | | |
| 82. | | | | | ═O | ═O |
| 83. | | | | | ═O | ═O |
| 84. | | | | | ═O | ═O |
| 85. | NHC₆H₅ | | | | ═O | ═O |
| 86. | | | | | ═O | ═O |
| 87. | | | | | ═O | ═O |
| 88. | | | | | ═O | ═O |
| 89. | | | | | ═O | ═O |
| 90. | NH₂ | | | | ═O | ═O |
| 91. | | | | | OH | OH |
| 92. | OH | | | | OH | OH |
| 93. | | | | | ═O | ═O |
| 94. | | | | | ═O | ═O |
| 95. | | | | | ═O | ═O |
| 96. | | | | | ═O | ═O |
| 97. | NH₂ | | | | ═O | ═O |
| 98. | | | | | OH | OH |
| 99. | OH | | | | OH | OH |
| 100. | OH | | | | OH | OH |
| 101. | OH | OH | | OH | ═O | ═O |
| 102. | 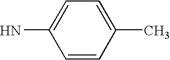 | | 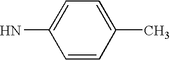 | | ═O | ═O |
| 103. | | | | | ═O | ═O |
| 104. | | | | | ═O | ═O |
| 105. | | | | | ═O | ═O |
| 106. | | | | | ═O | ═O |
| 107. | OH | OH | | | ═O | ═O |
| 108. | | | | | ═O | ═O |
| 109. | | | | | ═O | ═O |
| 110. | OH | OH | | 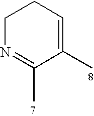 | ═O | ═O |
| 111. | OH | OH | | | ═O | ═O |
| 112. | NH₂ | OH | | | ═O | ═O |
| 113. | OH | OH | | OH | ═O | ═O |
| 114. | | | | | ═O | ═O |
| 115. | | | | | ═O | ═O |
| 116. | OH | | OH | | ═O | ═O |
| 117. | | | | p-NHC₆H₄CH₃ | ═O | ═O |
| 118. | | | | | ═O | ═O |
| 119. | | | | | ═O | ═O |
| 120. | | | | | ═O | ═O |
| 121. | | | | | ═O | ═O |
| 122. | | | | | ═O | ═O |
| 123. | | | | | ═O | ═O |
| 124. | | | | | ═O | ═O |
| 125. | | | | | ═O | ═O |
| 126. | | | | | ═O | ═O |
| 127. | | | | | ═O | ═O |
| 128. | | | | | ═O | ═O |
| 129. | | OH | | | ═O | ═O |
| 130. | NH₂ | | OH | | ═O | ═O |
| 131. | OH | | NH₂ | | ═O | ═O |
| 132. | | | | | ═O | ═O |
| 133. | | | | | ═O | ═O |
| 134. | NH₂ | SO₃H | OH | | ═O | ═O |
| 135. | NH₂ | | OH | | ═O | ═O |
| 136. | NH₂ | | | OH | ═O | ═O |
| 137. | | | | | ═O | ═O |
| 138. | NH₂ | | | | ═O | ═O |
| 139. | OH | | | | ═O | ═O |
| 140. | NH₂ | | | | ═O | ═O |
| 141. | NH₂ | | | | ═O | ═O |
| 142. | | | | | ═O | ═O |
| 143. | | | | | ═O | ═O |
| 144. | | | | | ═O | ═O |
| 145. | | | | | ═O | ═O |
| 146. | | | | | ═O | ═O |
| 147. | | | | | ═O | ═O |
| 148. | NH₂ | | SO₃H | OH | ═O | ═O |
| 149. | NHC₆H₅ | OH | | | ═O | ═O |
| 150. | NHC₆H₅ | OH | | | ═O | ═O |
| 151. | | OH | | | ═O | ═O |
| 152. | N(CH₃)₂ | OH | | | ═O | ═O |
| 153. | NHCOC₆H₅ | OH | | | ═O | ═O |
| 154. | | | | | ═O | ═O |
| 155. | | | | OH | ═O | ═O |
| 156. | | | | | ═O | ═O |

Dyes 1-39, 41-44, 46-50, 52-64, 66-80 and 82-156 are of the formula

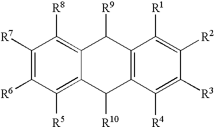

The examples 3, 39 and 40 are comparative examples and are not according to the invention.

### Example 2

Example 1 is repeated using the equivalent amount of any one of Dyes 2 to 156 in place of Dye 1.

Table 2 shows the results of photovoltaic cells made up using specific dyes according to Example 1. All results obtained are using Lil₂ propylene carbonate electrolyte in the cell.

TABLE 2

| | Photo- | Cell | | Conversion | |
|---|---|---|---|---|---|
| Example | Current | potential | Fill Factor | Efficiency | Intensity |
| No. | (mA/cm²) | (V) | (%) | (%) | W/m² |
| 3 | 0.72 | 0.29 | 0.61 | 1.43 | 89 |
| 3 | 7.10 | 0.34 | 0.41 | 1.11 | 890 |
| 5 | 0.55 | 0.37 | 0.60 | 1.80 | 70 |
| 5 | 5.30 | 0.43 | 0.53 | 1.73 | 700 |
| 1 | 0.95 | 0.37 | 0.61 | 2.50 | 78 |
| 1 | 9.00 | 0.43 | 0.60 | 3.10 | 750 |
| 6 | 0.54 | 0.38 | 0.70 | 2.05 | 70 |
| 6 | 5.20 | 0.45 | 0.60 | 2.10 | 670 |
| 101 | 0.36 | 0.47 | 0.51 | 0.13 | 870 |
| 102 | 0.78 | 0.55 | 0.63 | 0.61 | 880 |
| 103 | 0.20 | 0.32 | 0.31 | 0.39 | 910 |

### Example 3

Example 1 can be repeated using transparent TiO₂ film from colloidal titanium dioxide particles which are deposited on a conducting glass support and sintered to yield a coherent highly porous semiconducting film that is translucent instead of the 11th layer film in Example 1.

Colloidal titanium oxide particles of approximately 10 nm are prepared by hydrolysis of titanium isopropoxide as follows:

1 ml of titanium isopropoxide is added to a solution of 0.2M nitric acid in 100 ml of water whilst stirring. A precipitate of amorphous titanium dioxide is formed under these conditions. This is heated to 80° C. for approximately 8 hours resulting in peptisation of the precipitate and formation of a clear solution of colloidal anatase. The anatase structure of the titanium dioxide particles is established by Raman spectroscopy. The sol is concentrated by evaporation of the solvent in vacuum at room temperature until a viscous liquid is obtained containing the colloidal particles. At this stage the nonionic surfactant TRITON X-100 (20% volume) is added in order to stabilize the sol. The addition of the surfactant renders it possible to prepare TiO₂ sols having a solids content of 30-50 weight percent.

The titanium dioxide films are formed by spin coating the concentrated sol onto a conducting glass substrate. Usually it is sufficient to apply two or three layers in order to obtain semiconductor membranes of sufficient surface area to give excellent visible light harvesting efficiencies after deposition of a monolayer of the sensitizer.

The morphology of the films is examined by SEM, X-ray diffraction transmission spectroscopy and BET analysis of N₂ adsorption measured by a surface acoustic wave technique. Low resolution electron microscopy confirms the presence of the three layer structure, the lowest being the glass support followed by the 0.5 micron thick fluorine-doped SnO₂ and the 2.7 micron thick titanium dioxide layer. High resolution electron microscopy reveals the TiO₂ film to be composed of a three dimensional network of interconnected particles having an average size of approximately 16 nm. Apparently, significant particle growth occurs during sintering.

The transparent TiO₂ film and dye No. 1 of Table 1 is applied to produce a regeneration cell for the generation of electricity.

### Example 4

Example 3 can be repeated using instead of Dye 1 an equivalent amount of any one of dyes 2 to 156 of Table 1.

### BRIEF DESCRIPTION OF THE DRAWINGS

FIG. 1 represents an embodiment of the photovoltaic cell of the present invention.

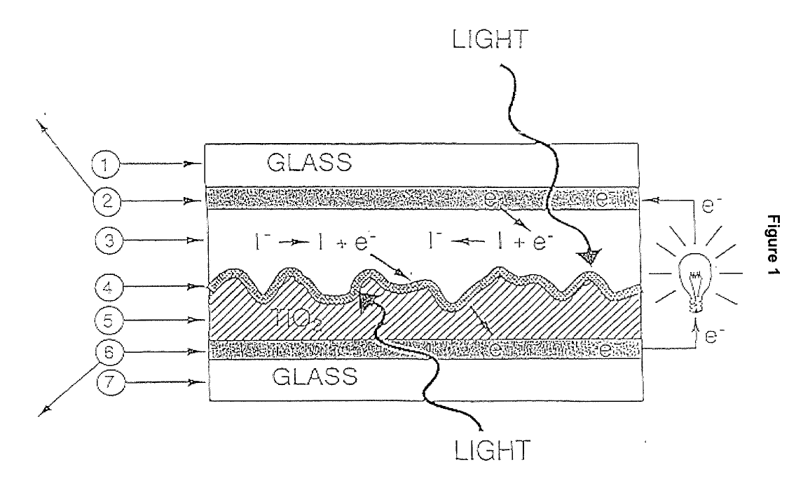

1. A dye-sensitized photochemical solar cell comprising dyes of the formula I, II, III. IV, V or VI 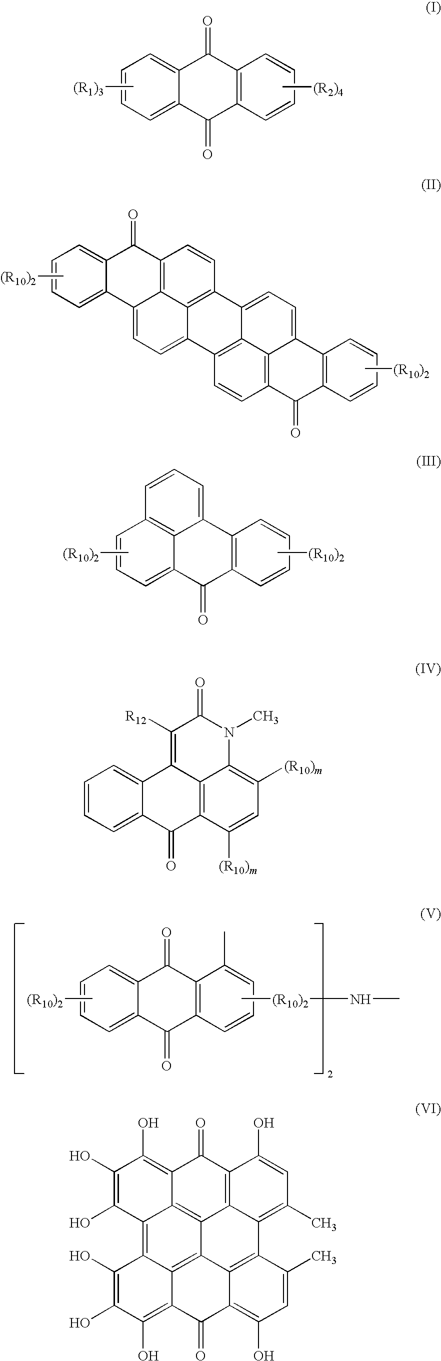
wherein each R₁ and R₂, independently is selected from the group consisting of hydrogen, —NH₂, —SO₃H, —SH, C₁₋₈ alkyl, —OH, —COOH, halogen, —NHC₁₋₄alkyl, —NH(CH₂)₁₋₂COOH, —NHCOR₃, —NHOH, —NHCH₂(CH₂)₁₋₂OH, —N(C₁₋₄alkyl)₂, 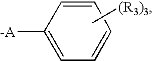
—OC₁₋₄alkyl, —OCH₂(CH₂)₁₋₂—COOH and —OCH₂(CH₂)₁₋₃—OH;
provided at least one group R₂ is hydrogen or two groups R₂ are ortho to one another and are the same as R₁ and the other two groups R₂ are ortho to one another and form a group α or β 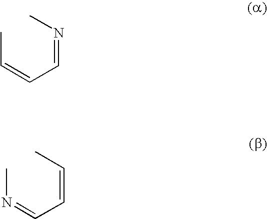
wherein
R₃ is selected from the group consisting of halogen C₁₋₄alkyl, —COOH, NH₂, OH and hydrogen;
A is —NH— or —O—;
R₁₀ is hydrogen, —NH₂, —OH, SH, —CO₂R₁₂, C₁₋₈alkyl, —(CH₂)₁₋₂—CO₂R₁₂, —NHR₁₂, —NR₁₂, —OR₁₂, —SR₁₂, where R₁₂ is hydrogen or C₁₋₈alkyl;
and m is 0 or 1 with the proviso that 1,2-dihydroxyanthra-9,10-chinone, 1,2,4-trihydroxyanthra-9,10-chinone and Isoviolanthrone are excluded.

2. A dye-sensitized photochemical solar cell according to claim 1 wherein the dye is selected from a compounds of formula I′ 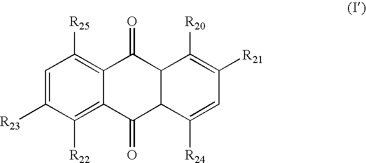
wherein each of R₂₀ to R₂₅ independently is hydrogen, —NH₂, OH, C₁₋₈ alkyl, 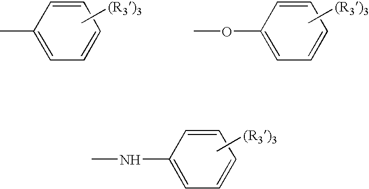
wherein R₃′ is hydrogen or C₁₋₄alkyl with the proviso that 1,2-dihydroxyanthra-9,10-chinone, and 1,2,4-trihydroxyanthra-9,10-chinone are excluded.

3. A photosensitizer dye in a metal oxide layer of a photovoltaic cell wherein in that the dye is one or more compounds of formula I to VI 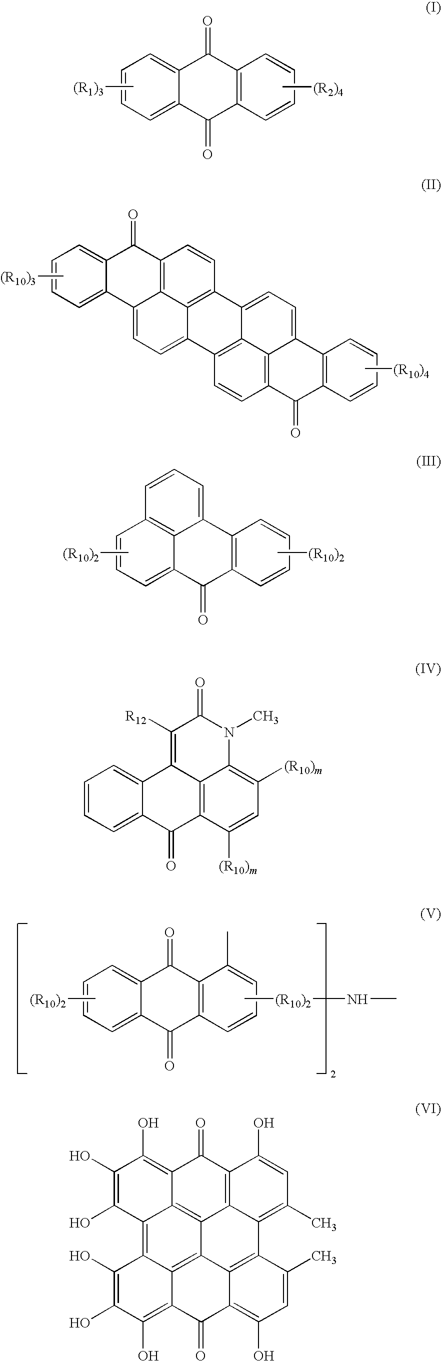
wherein each R₁ and R₂, independently is selected from the group consisting of hydrogen, —NH₂, —SO₃H, —SH, C₁₋₈ alkyl, —OH, —COOH, halogen, —NHC₁₋₄alkyl, —NH(CH₂)₁₋₂COOH, —NHCOR₃, —NHOH, —NHCH₂(CH₂)₁₋₂OH, —N(C₁₋₄alkyl)₂, 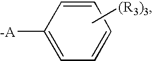
—OC₁₋₄alkyl, —OCH₂(CH₂)₁₋₂—COOH and —OCH₂(CH₂)₁₋₃—OH;
provided at least one group R₂ is hydrogen or two groups R₂ are ortho to one another and are the same as R₁ and the other two groups R₂ are ortho to one another and form a group α or β 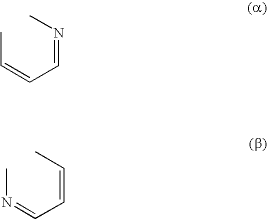
R₃ is selected from the group consisting of halogen C₁₋₄alkyl, —COOH, NH₂, OH and hydrogen;
A is —NH— or —O—;
R₁₀ is hydrogen, —NH₂, —OH, SH, —CO₂R₁₂, C₁₋₈alkyl, —(CH₂)₁₋₂—CO₂R₁₂, —NHR₁₂, —NR₁₂, —OR₁₂, —SR₁₂, where R₁₂ is hydrogen or C₁₋₈alkyl;
and m is 0 or 1
with the proviso that 1,2-dihydroxyanthra-9,10-chinone and 1,2,4-trihydroxyanthra-9,10-chinone and Isoviolanthrone are excluded.

4. The dye-sensitized photochemical solar cell according to claim 1, two groups R₂ are ortho to one another and are the same as R₁ and wherein R₁ is OH and the other two groups R₂ are ortho to one another and form a group α or β.

5. The photosensitizer dye in a metal oxide layer of a photovoltaic cell according to claim 3, wherein two groups R₂ are ortho to one another and are the same as R₁ and wherein R₁ is OH and the other two groups R₂ are ortho to one another and form a group α or β.

6. The photosensitizer dye in a metal oxide layer of a photovoltaic cell according to claim 3, wherein the dye is selected from a compounds of formula I′ 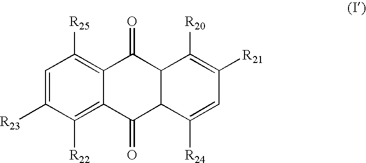
wherein each of R₂₀ to R₂₅ independently is hydrogen, —NH₂, OH, C₁₋₈ alkyl, 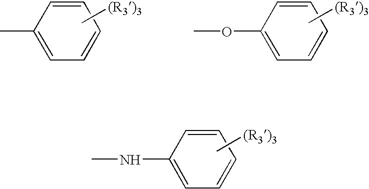
wherein R₃′ is hydrogen or C₁₋₄alkyl with the proviso that 1,2-dihydroxyanthra-9,10-chinone and 1,2,4-trihydroxyanthra-9,10-chinone are excluded.
